(19) United States
(12) Patent Application Publication
(10) Pub. No.: US 2025/0104875 A1
(43) Pub. Date: Mar. 27, 2025

(54) METHOD OF PREDICTING METASTASIS SITES

(71) Applicant: Siemens Healthineers AG, Forchheim (DE)

(72) Inventors: Svenja LIPPOK, Uttenreuth (DE); Sven KOHLE, Erlangen (DE); Ricky Anupam SHARMA, Lightwater (GB); Marta VILALTA, Belmont, CA (US)

(73) Assignee: Siemens Healthineers AG, Forchheim (DE)

(21) Appl. No.: 18/892,618

(22) Filed: Sep. 23, 2024

(30) Foreign Application Priority Data
Sep. 25, 2023 (DE) 10 2023 209 340.5

(51) Int. Cl. G16H 50/30; G16H 15/00; G16H 30/40; G16H 50/70

(52) U.S. Cl. CPC G16H 50/30; G16H 15/00; G16H 30/40; G16H 50/70

(57) ABSTRACT

One or more example embodiments provides a computer-implemented method of predicting secondary sites of a primary tumour. The method includes obtaining a whole-slide image of a primary tumour of a patient; obtaining a primary site descriptor of the primary tumour of the patient; inputting the whole-slide image and the primary site descriptor to a deep learning system previously trained to predict a metastasis probability for an appearance of a secondary tumour for one or more body parts of patients based on a particular whole-slide image and an associated particular primary site descriptor of a particular primary tumour; and outputting a secondary site prediction for at least one body part of the patient, wherein a secondary site prediction comprises a descriptor of the at least one body part and the metastasis probability associated with the at least one body part.

### CROSS-REFERENCE TO RELATED APPLICATION(S)

The present application claims priority under 35 U.S.C. § 119 to German Patent Application No. 10 2023 209 340.5, filed Sep. 25, 2023, the entire contents of which are incorporated herein by reference.

### Related Art

Metastases are the primary cause of cancer-related deaths, and intensive effort has been invested in understanding the mechanism of metastasis. The nature of metastasis is largely based on the characteristics of cancer cells. The ability to estimate, with some degree of certainty, the likelihood that a primary cancer will undergo metastasis already gives the treating physician valuable information that can help in deciding on a treatment plan for the individual patient.

However, depending on the nature of the primary tumour, secondary sites can establish at various different body regions. For example, circulating lung cancer cells can establish metastases in the bone, in the brain, etc.; ovarian cancer cells can establish metastases in the lung, in the liver, etc. Therefore, until metastases at such secondary sites have been identified in follow-up examinations, the treatment plan cannot include steps to target the specific types of metastases, and an estimation of where metastases may develop depends largely on the experience of the treating physician. To this end, the treating practitioner may schedule a biopsy (to obtain cancer cells from the primary tumour) and laboratory analytics to obtain data regarding the genomics, proteomics, transcriptomics etc. (referred to collectively as “omics”) of the primary tumour cells. However, while the prior art approach can be used to determine whether or not a primary tumour will at some point undergo metastasis, the treating physician must rely on experience in order to detect the actual metastases (“mets”) after these have already started to develop at one or more secondary sites.

### SUMMARY

A problem with this approach is that metastases are initially very small and therefore difficult to detect, and can establish at any number of essentially random locations. The delay in detecting metastases and the corresponding delay in commencing treatment is generally detrimental to the patient's outcome.

One or more example embodiments provides an improved way of evaluating information pertaining to a primary tumour.

### BRIEF DESCRIPTION OF THE DRAWINGS

Features of example embodiments will become apparent from the following detailed descriptions considered in conjunction with the accompanying drawings. It is to be understood, however, that the drawings are designed solely for the purposes of illustration and not as a definition of the limits of the invention.

FIG. 1 is a schematic diagram to illustrate a prior art approach according to one or more example embodiments;

FIG. 2 is a simplified block diagram to illustrate an inventive data processing system according to one or more example embodiments;

FIG. 3 shows an exemplary flowchart to illustrate an inventive method according to one or more example embodiments;

FIG. 4 shows a further schematic to illustrate an inventive data processing system according to one or more example embodiments; and

FIG. 5 illustrates application of one or more example embodiments during treatment of a patient.

In the diagrams, like numbers refer to like objects throughout. Objects in the diagrams are not necessarily drawn to scale.

### DETAILED DESCRIPTION

According to embodiments of the invention, the computer-implemented method comprises steps of obtaining a whole-slide image taken from a biopsy of a primary tumour in a patient's body; obtaining a descriptor of the primary site; inputting the whole-slide image and the primary site descriptor to a deep learning system (DLS) previously trained to identify the incidental metastasis probability, one or more body parts in which metastases of a primary tumour are likely to appear, and also the probability of metastases development at each identified body part; and outputting to a user a secondary site prediction for each identified body part of that patient. Each such secondary site prediction comprises a descriptor of the respective body part and the associated probability of metastases development at that body part (“secondary metastasis probability” or just “metastasis probability”). The incidental metastasis probability may also be output to the user.

According to embodiments of the invention, a computer-implemented method of predicting secondary sites of a primary tumour comprises: obtaining a whole-slide image of a primary tumour of a patient, obtaining a descriptor of the primary site of the primary tumour, inputting the whole-slide image and the primary site descriptor to a deep learning system previously trained to respectively predict a (secondary) metastasis probability for one or more body parts of a patient based on a whole-slide image and an associated primary site descriptor of a primary tumour, a (secondary) metastasis probability indicating a likelihood for an appearance of a secondary tumour of the primary tumour in an individual body part of the one or more body parts, and outputting a secondary site prediction for at least one body part of the patient, wherein the secondary site prediction comprises a descriptor of the at least one body part (secondary site) and the (secondary) metastasis probability associated with the at least one body part.

Whole-slide images may be two-dimensional digital images having a plurality of pixels. Whole slide images may have a size of at least 4.000×4.000 pixels, or at least 10.000×10.000 pixels, or at least 1E6×1E6 pixels.

A whole-slide image may image a tissue slice or slide of a patient. The preparation of the tissue slices from the tissue samples can comprise the preparation of a section from the tissue sample (for example with a punch tool), with the section being cut into micrometer-thick slices, the tissue slices. Another word for section is block or punch biopsy. Under microscopic observation, a tissue slice can show the fine tissue structure of the tissue sample and, in particular, the cell structure or the cells contained in the tissue sample. When observed on a greater length scale, a whole-slide image can show an overview of the tissue structure and tissue density. The tissue may have been taken from a tumour the patient is suffering from. In particular, the tissue may show a manifestation of a cancerous disease of the patient, such cells of a tumour.

The preparation of a tissue slice further may comprise the staining of the tissue slice with a histopathological staining. The staining in this case can serve to highlight different structures in the tissue slice, such as, e.g., cell walls or cell nuclei, or to test a medical indication, such as, e.g., a cell proliferation level. Different histopathological stains are used for different purposes in such cases.

To create the whole-slide image, the stained tissue slices are digitized or scanned. To this end, the tissue slices are scanned with a suitable digitizing station, such as, for example, a whole-slide scanner, which preferably scans the entire tissue slice mounted on an object carrier and converts it into a pixel image. In order to preserve the color effect from the histopathological staining, the pixel images are preferably color pixel images. Since in the prediction both the overall impression of the tissue and also the finely resolved cell structure may be of significance, the whole slide images typically have a very high pixel resolution. The data size of an individual image can typically amount to several gigabytes.

The descriptor of the primary site (“primary site descriptor” in the following) shall be understood as information that identifies the site of the primary tumour, and can be in the form of a text label, body coordinates, or other suitable annotation, added for example to a whole body image to specify the organ or organ sub-region in which the primary tumour is located. The same applies to the secondary site descriptor.

The one or more body parts may comprise organs or body compartments of the patient. A compartment may generally relate to an entity of the patient's organism. For instance, a compartment may relate to an organ, an organ part, an organ function, an anatomic structure, an anatomy, a functional unit of the patient's organism and so forth.

In particular, the deep learning system may be trained to predict the (secondary) metastasis probability for each of a plurality of different body parts of a patient. The plurality of different body part may be pre-determined. The plurality of different body parts may comprise organs of a patient such as the liver, the lung, the lymphatic system, the brain and so forth. In particular, the deep learning system may be trained to identify one or more body parts in which metastases of a primary tumour are likely to appear, and also the probability of metastases development at each identified body part.

The one or more body parts may be different from the body part the biopsy was taken from.

Outputting may comprise outputting a secondary site prediction for each of the plurality of body parts or for each of a subset of the plurality of body parts. In particular, outputting may comprise outputting a secondary site prediction for each of the plurality of body parts the (secondary) metastasis probability of which exceeds a predetermined threshold.

When a primary tumour undergoes metastasis, circulating tumour cells can be transported to various other locations (organs, body parts, body compartments, etc.) and the same type of tumour can start to develop at such new pathological sites or outlier locations. The location at which a metastasis appears is generally referred to as a “secondary site”. In contrast to the prior art approach of estimating metastasis development only from omics data, in the inventive method, the morphologic characteristics of the primary tumour cells in a whole-slide image are considered in view of the primary tumour site to derive a metastasis prediction specifically for individual body parts of the patient. An advantage of the inventive computer-implemented method is that it gives the user-a practitioner such as an oncologist, a radiologist, etc.-valuable information concerning the most likely future development of the primary tumour, assisting the practitioner in deciding on a subsequent monitoring plan even before the primary tumour undergoes metastasis. In this way, by monitoring the most likely secondary site locations, any metastases at such locations can be identified and treated at a very early stage. Another advantage is that biopsies and the ensuing pathological review is a standard procedure for cancer cases with a (digital) pathology examination being ordered in 95% of the cancer cases. This contrasts with sequencing tests which are ordered in considerably fewer numbers. Thus, the inventive method makes use of data which is readily obtained and can therefore integrate very well in an established clinical routine without entailing extra diagnostic costs and efforts.

According to some examples, the deep learning system is configured to determine an incidental metastasis probability of a primary tumour from a whole-slide image and/or a primary site descriptor of the primary tumour, and predict the (secondary) metastasis probability associated with one or more body parts based on the incidental metastasis probability and the primary site descriptor.

In particular, the inventors have recognized that a combination of a whole-slide image of a primary tumour and a location of the primary tumour in the patient's body provides a good basis for predicting further locations in the patient's body where secondary tumours of the primary tumour are likely to appear. Thereby, the whole-slide images might provide information about an “aggressivity” of the primary tumour while its location indicates which body parts are prone to be affected, for instance, due to a proximity to the primary tumour itself and/or to typical metastasis pathways like blood vessels or the lymphatic system. For the same reason, the location of the primary tumour may also affect the incidental metastasis probability. Accordingly, according to some examples, the primary site descriptor might also be considered for predicting the incidental metastasis probability.

As an alternative to the usage of a deep learning system, the metastasis prediction may be rule-based, for instance, involving the usage of medical guidelines which may set out metastasis pathways. In turn, the deep learning system may also be trained based on such medical guidelines.

As a further alternative, a similar patient search module may be provided which is configured to search for similar patients to the patient under consideration. The similar patients' cases may then be searched for documentations of metastasis. For the similar patient search the patient data and, optional, the incidental metastasis probability may be used as query vectors to a patient database such as an electronic medical record system.

According to some examples, the method further comprises generating a body model of the patient and registering the primary site and any secondary site with the body model, wherein the step of outputting comprises generating a rendering of the body model with the primary site, any secondary site and the corresponding (secondary) metastasis probability highlighted.

According to some examples, the body model may be generated based on basic health and demographic data of the patient such gender, age, weight, height and so forth. The basic health and demographic data may be obtained from the electronic medical record of the patient. Generating the body model may comprise adapting a standard body model based on the basic health and demographic data.

Registering the site descriptors may comprise determining a location of the sites in the body model.

Highlighting may mean that the primary site (location), the secondary site (location) and the (secondary) metastasis probability are visually conceivable for a user in the rendering. For instance, the (secondary) metastasis probability may be highlighted by inserting numbers reflecting the (secondary) metastasis probability in the rendering, such as a percentage. In addition or as an alternative, the (secondary) metastasis probability may be highlighted in the form of a colour coding.

According to one or more example embodiments, a data processing system adapted to carry out such a computer-implemented method comprises an input interface for receiving a whole-slide image of a primary tumour of a patient and a site descriptor of the primary tumour; a deep learning system previously trained to determine the incidental metastasis probability of a primary tumour from a whole-slide image and primary site descriptor, to identify any body part at which a secondary tumour is likely to appear, and to predict the (secondary) metastasis probability associated with each identified body part; and an output interface for outputting a secondary site prediction for each identified body part of that patient, wherein a secondary site prediction comprises a descriptor of the body part and the (secondary) metastasis probability associated with that body part.

One or more example embodiments further describes a computer program product comprising instructions which, when the program is executed by a processor of such a data processing system, cause the data processing system to carry out the steps of the inventive method.

The units or modules of the data processing system mentioned above, in particular the deep learning system, can be completely or partially realised as software modules running on a processor of the data processing system. A realisation largely in the form of software modules can have the advantage that applications already installed on an existing system can be updated, with relatively little effort, to install and run the computer program product of the present application. In addition to the computer program, such a computer program product can also comprise further parts such as documentation and/or additional components, also hardware components such as a hardware key (dongle etc.) to facilitate access to the software.

A computer readable medium such as a memory stick, a hard-disk or other transportable or permanently-installed carrier can serve to transport and/or to store the executable parts of the computer program product so that these can be read from a processor of a data processing system. A processor unit can comprise one or more microprocessors or their equivalents.

Particularly advantageous embodiments and features of the invention are given by the dependent claims, as revealed in the following description. Features of different claim categories may be combined as appropriate to give further embodiments not described herein.

The inventive data processing system comprises a first stage in which the whole-slide image is analysed to determine the incidental probability of metastasis, and a second stage in which potential secondary site locations are predicted, along with the probability of metastases developing at those secondary site locations. In the first stage of the data processing system, the deep learning system preferably comprises a first machine learning algorithm previously trained to predict the incidental metastasis probability of the primary tumour based on the whole-slide image alone, i.e., the incidental metastasis probability is derived from the morphologic characteristics of primary tumour cells shown in the whole-slide image. This first machine learning algorithm can be a residual network (“ResNet”), for example, trained to extract image data from the whole-slide image and to convert image data into feature vectors. To improve the image processing, the whole-slide image can be divided into an array or matrix of “patches”, and a feature vector can be extracted for each patch. The set of features can then be averaged to obtain an average feature vector for the whole-slide image. Such a machine learning algorithm can be an off-the-shelf product already available for use by medical image processing tools, for example.

In the second stage of the data processing system, the deep learning system preferably comprises a prediction module previously trained to determine the potential secondary sites in the patient's body and the probability of metastases at each secondary site, based on the primary site descriptor and the incidental metastasis probability. The prediction module can for example be a regressor model, preferably based on gradient boosting.

The inventive deep learning system is preferably trained using data from a cohort of patients to predict the occurrence of metastasis for distinct body parts based on the knowledge of a primary cancerous growth. A training dataset for the deep learning system of the inventive data processing system preferably comprises at least one whole image slide of a primary tumour, a primary site descriptor, and information regarding the occurrence of metastasis of the primary tumour for that patient. Preferably, the deep learning system is trained using many such training datasets, for example several thousand datasets. The training datasets can be obtained from one or more picture archiving and communication systems (PACS), for example.

Since the incidental metastasis probability is determined from features identified in a whole-slide image in the first stage of the data processing system, this part of the deep learning system can be referred to as the “image branch”, and the incidental metastasis probability can be regarded as an image-derived feature or vector or can be regarded as being encoded in such a feature or vector. In the second stage of the data processing system, the prediction of the secondary site metastases location(s) and their respective metastases probability can be performed on this vector alone.

The prediction of the secondary site metastases, or metastasis prediction for distinct body parts, shall be understood as the likelihood that metastases will develop at a specific secondary site. The outcome of the second stage can be in the form of a probability for each secondary site, e.g., 50% probability of metastases forming in the liver, 10% probability of metastases forming in the bowel.

With the metastasis prediction for distinct body compartments, the physician is provided with an actionable result. Using the information provided by the inventive data processing system, the practitioner can retrieve already existing (medical) diagnostic images from the patient's EMR and examine these in the light of the predicted secondary sites. Equally, the practitioner can schedule further radiology examinations in good time, to obtain images showing the identified secondary site locations. According to some examples, the (medical) diagnostic images may comprise one or more radiological medical images.

The radiological medical images may comprise one or more medical image data sets. A medical image data set may be a two-dimensional image. Further, the medical image data set may be a three-dimensional image. Further, the medical image may be a four-dimensional image, where there are three spatial and one time-like dimensions. Further, the medical image data set may comprise a plurality of individual medical images.

The medical image data set comprises image data, for example, in the form of a two-or three-dimensional array of pixels or voxels. Such arrays of pixels or voxels may be representative of color, intensity, absorption or other parameters as a function of two or three-dimensional position, and may, for example, be obtained by suitable processing of measurement signals obtained by a medical imaging modality or image scanning facility.

The medical image data set may be a radiology image data set depicting a body part of a patient. Accordingly, it may contain two or three-dimensional image data of the patient's body part. The medical image may be representative of an image volume or a cross-section through the image volume. The patient's body part may be comprised in the image volume.

A medical imaging modality corresponds to a system used to generate or produce medical image data. For example, a medical imaging modality may be a computed tomography system (CT system), a magnetic resonance system (MR system), an angiography (or C-arm X-ray) system, a positron-emission tomography system (PET system) or the like. Specifically, computed tomography is a widely used imaging method and makes use of “hard” X-rays produced and detected by a spatially rotating instrument. The resulting attenuation data (also referred to as raw data) is processed by a computed analytic software producing detailed images of the internal structure of the patient's body parts. The produced sets of images are called CT-scans which may constitute multiple series of sequential images to present the internal anatomical structures in cross sections perpendicular to the axis of the human body. Magnetic Resonance Imaging (MRI), to provide another example, is an advanced medical imaging technique which makes use of the effect magnetic field impacts on movements of protons. In MRI machines, the detectors are antennas, and the signals are analyzed by a computer creating detailed images of the internal structures in any section of the human body.

The medical image may be stored in a standard image format such as the Digital Imaging and Communications in Medicine (DICOM) format and in a memory or computer storage system such as a Picture Archiving and Communication System (PACS), a Radiology Information System (RIS), and the like. Whenever DICOM is mentioned herein, it shall be understood that this refers to the “Digital Imaging and Communications in Medicine” (DICOM) standard, for example according to the DICOM PS3.1 2020c standard (or any later or earlier version of said standard). The inventive data processing system can comprise a number of additional “non-image branches”. For example, in a further embodiment of the invention, the data processing system comprises a second branch and the DLS comprises a second machine learning algorithm previously trained to derive features from omics data. This second machine learning algorithm can be a convolutional neural network (CNN) previously trained to derive molecular markers from sequenced genomic data in this non-image branch. Such a trained machine learning algorithm can be an off-the-shelf product already available to the laboratory tasked with the analysis of the biopsy tissue, for example. Omics data can, for example, be obtained by high-throughput sequencing of specimens such as primary tumour biopsy material. The morphological cues obtained from the whole-slide image regarding the propensity of the primary tumour to undergo metastasis can then be augmented by insights from molecular analysis.

In such an embodiment, the prediction module is configured to determine a metastasis prediction on the image-derived features and also on the basis of the omics-derived features. A training dataset for the deep learning system therefore preferably also comprises any omics data that is relevant to the whole-image slide of the dataset. The additional omics data could be obtained from the clinic's electronic medical record (EMR) database, for example, since omics analytics are generally stored as part of the patient's EMR.

In a further embodiment of the invention, the data processing system comprises a further non-image branch, and the DLS comprises a third machine learning algorithm previously trained to derive features from data such as medical records, preferably EMRs available to the data processing system. It is known that factors such as age, co-morbidities, habits regarding diet, smoking, alcohol consumption etc., may affect the likelihood of metastasis occurring in distinct body parts, and such information may be stored in the patient's EMR. Further information on record can comprise numerical entries, screenshots, patient-related data such as age, sex, etc. This third machine learning algorithm can be a large language model (LLM) based on a transformer architecture. Such a trained machine learning algorithm can be an off-the-shelf product already available for the purpose of extracting information from an EMR database, for example. Furthermore, if a recent report indicates that there are no findings for a specific body part, this may be indicated to the practitioner. Equally, the likelihood of a metastasis in this body part may be corrected on the basis of the output of the deep learning system.

In such an embodiment, the prediction module is configured to determine a metastasis prediction on the image-derived features and also on the basis of the records-derived features, and may also base its prediction on the omics-derived features mentioned above. A training dataset for the deep learning system therefore preferably also comprises the patient's EMR, obtained for example from the clinic's archiving system.

The image-derived features and optionally the records-derived features and/or the omics-derived features can be combined to obtain a concatenation vector as input to the second stage of the DPS or “prediction module”.

In a further embodiment of the invention, the output of the second stage can be in the form of a schematic body model of the patient, and each predicted location or body compartment can be highlighted according to the likelihood of metastasis at that location. The output of the prediction module also includes an estimation of the severity of metastases that may develop at the predicted locations.

In a further embodiment of the invention, the inventive method comprises an additional step of querying an image database (e.g., a local PACS database) for, in particular radiological, medical diagnostic images previously obtained for the patient, retrieving at least one image of the previously obtained radiological images based on the secondary site prediction (wherein the at least one image is showing the respective body part of the secondary site prediction), and providing the at least one retrieved image. In a further embodiment, the method includes the steps of subsequently identifying the type (e.g. MRI, CT) of each retrieved image, and deploying a suitable algorithm for analysing that image type to identify metastases present in the retrieved image. An algorithm such as a segmentation algorithm is generally tailored to a specific type of medical image, for example image processing of a CT image can depend on the image re-construction parameters used to create the image. In this way, hitherto undetected metastases may be discovered in a retrieved image. In a further embodiment, the inventive method comprises an additional step of informing the corresponding practitioner (the doctor or clinician that originally ordered the diagnostic image) of the newly detected metastases.

According to some examples, querying may be based on an electronic patient identifier with which the patient is registered in the image database. Further, querying may be based on metadata respectively associated with the medical diagnostic images indicating the body part depicted by the associated medical diagnostic image. For instance, such metadata may comprise the DICOM-headers of the respective medical diagnostic images. According to some examples, querying may be limited to “recent” medical diagnostic images which can provide insights into the progression of the cancerous disease of the patient. For instance, recent may mean a time period of one, two, or three months around the point in time when a biopsy for preparing the whole-slide image was done and/or any time period after the point in time when a biopsy for preparing the whole-slide image was done.

According to some examples, the method may further comprise indicating medical diagnostic images comprised in the image database in the body model according to the body parts respectively depicted. Specifically, it may be indicated in the body model that a medical diagnostic image is available for a certain body part. With that, a user may get an immediate overview about the available information. On that basis, the user can selectively call-up medical diagnostic images or infer at one glance where medical diagnostic image information is missing to assess possible metastases.

The inventive data processing system is preferably also configured to determine whether all relevant body compartments (body parts at which metastases are likely to develop) are covered by the images retrieved from the medical image database. If not, the data processing system informs the user of any missing diagnostic images, for example by an appropriate annotation to a body model.

Equally, the inventive method can comprise a step of proposing a radiology examination for an identified secondary site location for which a recent diagnostic image is not available. This function can include a step of selecting imaging parameters with which the relevant medical imaging modality should be controlled (e.g. contrast agent administration, MRI pulse sequences etc.) in order to obtain an optimal image showing a predicted secondary site. This function can also include a step of automatically scheduling the corresponding imaging and examination task(s).

Specifically, according to an embodiment, the method may further comprise, if the image database does not comprise medical diagnostic images showing the at least one body part: generating a medical imaging protocol which is suited to generate a medical diagnostic image showing the at least one body part, and outputting the medical imaging protocol.

The imaging protocol may comprise human-readable and/or machine readable instructions. For instance, the instructions may comprise steps and/or parameters for performing the medical examination directed to obtain the medical diagnostic image showing the at least one body part.

According to some examples, the imaging protocol comprises machine-readable instructions configured to control a modality configured to perform a medical examination for obtaining the medical diagnostic image showing the at least one body part and the method further comprises: inputting/forwarding the imaging protocol in/to the modality (so as control the modality to perform the medical examination), and/or providing the instruction to a user via a user interface (for review and further usage).

According to some examples, the method further comprises retrieving a medical diagnostic image of the patient showing the at least one body part from an image database, applying a detection function to the retrieved medical diagnostic image so as to generate a detection result, the detection function being configured to detect tumours in medical diagnostic images, and outputting the detection result.

A detection function may generally be configured to detect medical findings or abnormalities, and, in particular, cancerous growths/tumours, in particular secondary tumours, in medical image data. In principle, a plethora of functionalities and methods is known for such computer aided detection and classification of abnormalities-all of which may be implemented in the abnormality detection algorithm. For instance, reference is made to US 2009/0 092 300 A1,US 2009/0 067 693 A1, or US 2016/0 321 427 A1, the contents of which are incorporated herein in their entirety by reference.

A detection result may comprise an indication of the presence of a finding, in particular, a (secondary) tumour, in the medical diagnostic image. According to some examples, the detection result may comprise a location of the (secondary) tumour and/or a type of the (secondary) tumour and/or one or more characteristics of the (secondary) tumour such as a size, a contour (e.g., level of spiculation or lobulation) or composition (e.g., level of calcification). Further, the detection result may comprise an indication that no finding/no secondary tumour was detected in the medical diagnostic image.

Outputting the detection result may comprise outputting the detection result to a user via a user interface. Further outputting the detection result may comprise indicating the detection result in the body model. The latter may comprise registering the detection result with the body model so as to determine a corresponding location of the detection result in the body model and highlighting the detection result in a rendering of the body model at the corresponding location.

In a further embodiment of the invention, the inventive method comprises an additional step of grading the incidental metastasis probability. To this end, the deep learning system can have been trained to also predict the severity of a secondary tumour based on the primary tumour type and location, and using other patient-related information such as the patient's age, smoking history, pre-existing conditions, etc.

In a further embodiment of the invention, the deep learning system can be configured to include a patient search module realized to search a database for medical records of patients similar to the patient under consideration. The similar patient cases may then be searched for documentations of metastasis. Data of the patient under consideration and/or the incidental metastasis probability may be used as a query vector for the similar patient search.

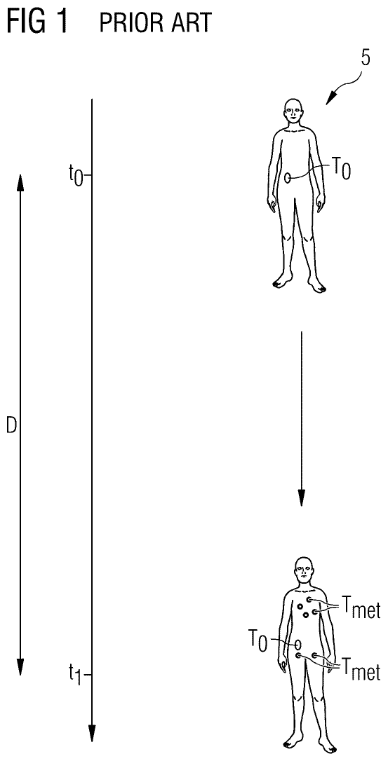

FIG. 1 is a schematic diagram to illustrate a prior art treatment progression for a patient 5 with a primary tumour T0 at a certain body location. The body location can largely define the nature of that primary tumour T0. In the upper part, the diagram indicates an early stage of cancer, i.e. prior to metastasis of the primary tumour T0, detected in a diagnostic image at time t0. For an oncologist treating this patient 5, it is important to monitor development of the cancer so that appropriate treatments can be scheduled in a timely manner. In some cases, a type of primary tumour can remain localized for a long time before undergoing metastasis, while the same type of tumour in another patient may undergo metastasis sooner. Once circulating cancer cells reach the bloodstream or lymphatic system, the next stage of cancer begins, and ultimately metastases (“mets” or secondary tumours) will appear at—usually multiple—secondary sites as indicated. At this stage of cancer, it is crucial to detect the secondary tumours as early as possible. In the past, the oncologist, radiologist and other practitioners have relied on their experience to identify the most suitable treatment plan for the patient. However, a prior art treatment plan can only be put together after detection of a secondary tumour, which in turn relies on the practitioner's timely scheduling of a radiology exam to obtain diagnostic images for the corresponding body part, for example at the considerably later time t1. The delay D between initial detection of the primary tumour at date to to detection of the metastases at date t1 in the prior art approach has generally had a negative effect on patient outcome.

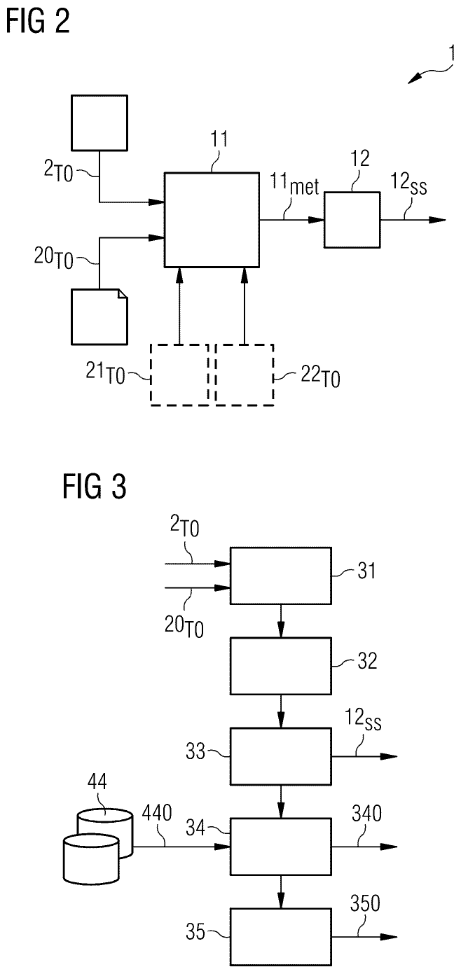

FIG. 2 is a simplified block diagram to illustrate the inventive data processing system 1, and FIG. 3 shows an exemplary flowchart to illustrate the inventive method. In a first step 31, a whole-slide image 2T0 of a primary tumour is obtained for a patient under consideration, as well as a site descriptor 20T0 for that primary tumour. The whole-slide image 2T0 shall be understood to show tissue obtained from a biopsy of a primary tumour T0. The whole-slide image 2T0 may be stained in the standard H&E stain. The primary site descriptor 20T0 can be any suitable label that clearly identifies the location of the primary tumour T0, for example the organ from which biopsy tissue was taken, the biopsy coordinates, etc.

Next, in step 32, the whole-slide image 2T0 and primary site descriptor 20T0 are input to a deep learning system 11, 12 which has been trained to predict one or more locations at which metastases are likely to develop.

The deep learning system comprises a first stage 11 in which the incidental metastasis probability 11met is predicted. This first stage comprises a machine learning algorithm that is trained to predict the incidental metastasis probability 11met from the whole-slide image 2T0 and the primary site descriptor 20T0, and may comprise further machine learning algorithms that make contributions to the prediction on the basis of omics data 21TO and/or EMR data 22T0 as described above. The output of this first stage 11 is the incidental metastasis probability 11met. The deep learning system 1 comprises a second stage 12 with a machine learning algorithm that is trained to predict any secondary site locations—i.e. body parts or body regions in which metastases of a primary tumour are likely to occur—as well as the probability of metastases development for each secondary site location from the incidental metastasis probability 11met. The secondary site prediction(s) 12SS are output to the user in a subsequent step 33. Each secondary site prediction 12SS comprises an identifier or descriptor of the predicted secondary site location as well as the probability of metastases development at that secondary site location. The predicted incidental metastasis probability 11met may also be output to the user.

In this exemplary embodiment, the workflow includes a step 34 of querying a database such as a PACS database 44 for medical images previously ordered for that patient. Any retrieved image can be analysed using a suitable algorithm as explained above, to inspect each image for hitherto undetected metastases. An appropriate report 340 can be issued to the user of the DPS 1, or to any practitioner that had ordered a diagnostic image in which a secondary site has now been discovered.

This can be followed by another step 35 in which a radiology workflow 350 for that patient is suggested to the practitioner in order to schedule imaging procedures to obtain images of the predicted locations at which secondary sites are likely to develop.

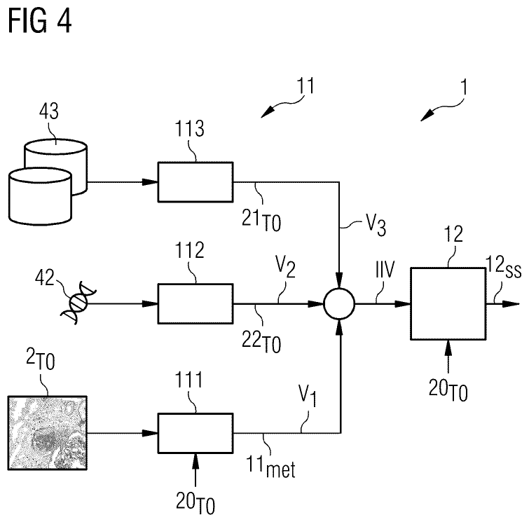

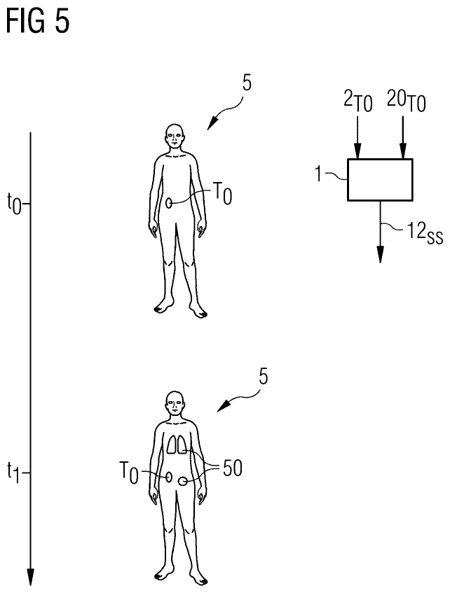

FIG. 4 shows a further block diagram to illustrate the inventive approach. Here, the data processing system 1 is shown to comprise an image branch and two optional non-image branches.

In the image branch, a whole-slide image 2T0 and site descriptor 20T0 are obtained as described above. A machine learning algorithm 111 such as a residual network (“ResNet”), trained to predict the incidental metastasis probability from a whole-slide image 2T0 and a primary site descriptor 20T0, returns the incidental metastasis probability 11met. Here, the incidental metastasis probability 11met can be regarded as an image-derived vector V1.

In one (optional) non-image branch, a machine learning algorithm 112 such as a LLM, trained to extract information from electronic medical records stored in an EMR database 42, returns records-derived features 22T0 as a records-derived vector V2. In the other (optional) non-image branch, a machine learning algorithm 113 such as a CNN, trained to extract information from omics data 43, returns omics-derived features 21TO as an omics-derived vector V3.

To make use of all this information, the image-derived vector V1, the records-derived vector V2 and the omics-derived vector V3 can be combined as shown here to give a concatenation vector 11V as input to the prediction module 12, which then returns its secondary site location prediction 12SS. Of course, the prediction module 12 can make its prediction based only on the image-derived vector V1, based on the image-derived vector V1 in combination with the omics-derived vector V3, or based on the image-derived vector Vlin combination with the records-derived vector V2.

FIG. 5 is a schematic diagram to illustrate application of one or more example embodiments during treatment of a patient 5 with a detected primary tumour T0. Again, the diagram indicates an early stage of cancer detected at time t0. A practitioner treating this patient 5 orders a biopsy to obtain a whole-slide image 2T0, and inputs this, along with a primary site descriptor 20T0, to an instance of the inventive data processing system 1, which returns one or more secondary site predictions 12SS. Each secondary site prediction 12SS comprises a descriptor of a body part 50 at which mets of this primary tumour T0 are likely to develop, and the probability of metastasis at that body part. Follow-up images of the patient 5 can then be scheduled for the near future, as indicated here at time t1, so that secondary tumours can be detected early at the predicted sites 50.

Equally, already existing images 440 showing those body regions can be retrieved from a medical image database 44 as described above and analysed to detect lesions that may be present but have gone undetected, in which case there is no delay until detection of the mets (t1=t0).

Since the secondary sites for likely mets can be identified even before metastasis of the primary tumour T0, one or more example embodiments essentially removes the unnecessary delay D that is common in the prior art approach, and can significantly improve patient outcome.

Although the present invention has been disclosed in the form of embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module. Wording in brackets indicates optional and/or clarifying features or wording. Independent of a grammatical term usage, individuals with male, female or other gender identities are included. Any pronoun denoting a specific gender shall be understood to apply equally to any gender identity.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. computer The programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method of predicting secondary sites of a primary tumour, the method comprising:
obtaining a whole-slide image of a primary tumour of a patient;
obtaining a primary site descriptor of the primary tumour of the patient;
inputting the whole-slide image and the primary site descriptor to a deep learning system previously trained to predict a metastasis probability for an appearance of a secondary tumour for one or more body parts of patients based on a particular whole-slide image and an associated particular primary site descriptor of a particular primary tumour; and
outputting a secondary site prediction for at least one body part of the patient, wherein a secondary site prediction comprises a descriptor of the at least one body part and the metastasis probability associated with the at least one body part.

2. The computer-implemented method of claim 1, wherein the deep learning system is configured to:
determine an incidental metastasis probability from at least one of the whole-slide image or the primary site descriptor of the primary tumour, and
predict the metastasis probability respectively associated with one or more body parts based on the incidental metastasis probability and the primary site descriptor.

3. The computer-implemented method of claim 1, wherein a secondary site prediction is further based on omics-derived features.

4. The computer-implemented method of claim 1, wherein a secondary site prediction is further based on records-derived features.

5. The computer-implemented method of claim 1, further comprising:
querying an image database for medical diagnostic images of the patient showing the at least one body part.

6. The computer-implemented method of claim 5, further comprising:
generating a medical imaging protocol suited to generate a medical diagnostic image showing the at least one body part if the image database does not comprise medical diagnostic images showing the at least one body part; and
outputting the medical imaging protocol.

7. The computer-implemented method of claim 1, further comprising:
retrieving a medical diagnostic image of the patient showing the at least one body part from an image database;
applying a detection function to the retrieved medical diagnostic image to generate a detection result, the detection function being configured to detect tumours in medical diagnostic images; and
outputting the detection result.

8. The computer-implemented method of claim 1, further comprising:
proposing a radiology examination for the at least one body part.

9. The computer-implemented method of claim 1, further comprising:
generating a body model of the patient; and
registering a primary site associated with the primary site descriptor and the at least one body part with the body model, wherein
the outputting comprises generating a rendering of the body model with a primary site location, the at least one body part, and the corresponding metastasis probability highlighted.

10. A data processing system configured to perform out the computer-implemented method of claim 1, the data processing system comprising:
an input interface configured to receive the whole-slide image and the primary site descriptor;
the deep learning system; and
an output interface configured to output the secondary site prediction for each identified body part of the patient.

11. The data processing system of claim 10, wherein the deep learning system comprises a first machine learning algorithm previously trained to predict the incidental metastasis probability of the primary tumour based on the whole-slide image.

12. The data processing system of claim 10, wherein the deep learning system comprises a prediction module previously trained to predict one or more metastasis locations based on the primary site descriptor and an incidental metastasis probability.

13. The data processing system of claim 12, comprising a second machine learning algorithm previously trained to derive omics features from omics data, and wherein the prediction module is configured to predict one or more metastasis locations based on the omics-derived features.

14. The data processing system of claim 13, wherein the second machine learning algorithm comprises a convolutional neural network trained to derive molecular markers from sequenced genomic data.

15. The data processing system of claim 10, comprising a third machine learning algorithm previously trained to derive features from medical records of that patient, and wherein the prediction module is configured to predict one or more metastasis locations based on the derived features from the medical records.

16. The data processing system of claim 15, wherein the third machine learning algorithm comprises a large language model based on a transformer architecture.

17. The data processing system of claim 10, wherein a training dataset for the deep learning system comprises:
at least one whole image slide image of a training primary tumour;
a training primary tumour site descriptor; and
training information regarding an occurrence of metastasis of the primary tumour.

18. A non-transitory computer program product comprising instructions which, when executed by a data processing system, cause the data processing system to perform the method of claim 1.

19. A non-transitory computer readable medium comprising instructions which, when executed by a data processing system, cause the data processing system to perform the method of claim 1.
